(19) United States
(12) Patent Application Publication
(10) Pub. No.: US 2013/0030383 A1
(43) Pub. Date: Jan. 31, 2013

(54) INJECTION DEVICE

(71) Applicant: Joachim Keitel, Esslingen (DE)

(72) Inventor: Joachim Keitel, Esslingen (DE)

(73) Assignee: HASELMEIER GMBH, STUTTGART, BW (DE)

(21) Appl. No.: 13/393,359

(22) PCT Filed: Sep. 21, 2010
(86) PCT No.: PCT/EP2010/005762

§ 371 Date: Oct. 15, 2012

(30) Foreign Application Priority Data
Sep. 26, 2009 (DE) 10 2009 048 497.3

(51) Int. Cl. A61M 5/315

(52) U.S. Cl. 604/232

(57) ABSTRACT

An injection device has a housing (34), and a carpule container (44) for receiving a carpule (50) having a fluid (52) to be injected and having a piston (48) displaceable in said carpule (50). It further has a piston rod (38) with end plate (46′) which serves, in the context of an injection, to displace the piston (48) of a carpule (50) inserted into the carpule container (44) and thereby to eject fluid (52) from the carpule (50). In order to minimize patient error in adjusting the amount of fluid medication (52) to be injected, the mechanism includes a first component (68) and a second component (92) which link (98) and interact with each other, to control when the carpule container (44) can rotate with respect to the housing (34), and to prevent creation of an axial gap (62) between end plate (46′) and piston (48).

### CROSS-REFERENCE TO RELATED APPLICATIONS

This application is a section 371 of PCT/EP10/05762, filed Sep. 9, 2010 published Mar. 31, 2011 as WO-2011-035 877-A2, and further claims priority from application DE 10 2009 048 497.3 filed Sep. 26, 2009 the entire disclosure of which is hereby incorporated by reference.

### FIELD OF THE INVENTION

The invention relates to an injection device that preferably serves to perform one or more injections, depending on the medication demand of the patient, from a medication reservoir that is also referred to as a “carpule”. The injections occur through an injection needle that can also be referred to as a “hollow needle.”

### BACKGROUND

A carpule has a displaceable piston, usually made of an elastomer, and this piston is also referred to as a “plunger.” Upon injection, the plunger is pushed forward, i.e. toward the patient, by a piston rod that is also referred to as a “dosing piston,” in order to expel medication through the injection needle.

In usual embodiments of such injection devices, the desired motion of the piston can be adjusted in 0.14-mm steps. It is thus necessary, in order to achieve the desired precision in terms of the quantity of medication injected, for the position of the piston at the beginning of an injection to be accurately known.

With many injection devices the carpule is replaceable, i.e. when the contents of a carpule are exhausted, a new one is inserted into the injection device. After insertion of a new carpule, the location of the piston in it is not accurately known. Even in the case of carpules that are filled automatically with a consistent volume, the location of the piston can fluctuate by approximately +/−0.5 mm.

In the case of a pen injector in which the carpule can be replaced, it is therefore necessary, after a carpule change, to “preset” the piston rod against the piston of the carpule, i.e. after presetting, a clearance must no longer exist between the piston rod and piston.

Presetting usually occurs by the fact that the patient, after inserting the carpule and after threading on an injection needle, repeatedly sets a small injection dose and performs “injections” into the air until the piston rod is resting snugly against the piston and medication emerges for the first time from the injection needle. This procedure is called “priming.” In practice, it is important that this operation be as simple and intuitive as possible, since it will otherwise easily be overlooked.

Pen injectors usually have a holder into which the carpule is inserted and which is mounted on the pen injector. This holder, which can also be referred to as a “carpule container,” is configured on its patient-side, i.e. front, end so that an injection needle can be attached, for example by means of a bayonet connection or a screw thread.

### SUMMARY OF THE INVENTION

It is therefore an object of the invention to make available a novel injection device whose structure minimizes any need for priming.

According to the invention, this object is achieved by structuring the injection mechanism with a linkage including a first component and a second component. The first component is arranged rotatable but not axially displaceable with respect to the housing. The second component is arranged non-rotatable but axially displaceable with respect to the piston rod. The first and second components interact in such a way that, once the end plate of the piston rod abuts against the piston, rotation of the carpule with respect to the housing is blocked, and no axial gap can be inadvertently created between the end plate and the piston.

### BRIEF FIGURE DESCRIPTION

Further details and advantageous refinements of the invention are evident from the exemplifying embodiments, in no way to be understood as a limitation, that are described and depicted below.

FIG. 1 shows an injection device 28 in the form of a so-called pen injector, viewed in the direction of arrow I of FIG. 2;

FIG. 2 shows the injection device of FIG. 1 viewed in the direction of arrow II of FIG. 1, the cartridge being partly filled with the medication;

FIG. 3 shows the injection device according to FIGS. 1 and 2, in which device the stock of medication is exhausted, and in which the empty carpule must therefore be replaced with a full carpule;

FIG. 4 shows the same injection device, in which the piston rod has been screwed back, by rotation of the carpule container in the direction of an arrow 58, in order to enable replacement of the empty carpule;

FIG. 5 shows the injection device of FIG. 4, in which device the carpule container (which still contains the empty carpule) has been removed;

FIG. 6 shows the injection device of FIG. 5, in which device the old carpule has been removed from the carpule container and substituted with a new one;

FIG. 7 shows the injection device of FIG. 6, in which device the carpule container is mounted on the housing of the injection device;

FIG. 8 shows the injection device of FIG. 7, in which device the piston rod has been brought, by rotation of the carpule container, against the piston in the new carpule and is abutting against it with no clearance;

FIG. 9 is a depiction analogous to FIG. 8 but at enlarged scale; this figure serves to explain that there must be no air gap present between the piston in the carpule and a plate mounted on the piston rod;

FIG. 10 is a side view of a carpule container 44, viewed in the direction of arrow X of FIG. 11;

FIG. 11 is a longitudinal section viewed along line XI-XI of FIG. 10, a filled carpule 50 being located in carpule container 44;

FIG. 12 is a three-dimensional depiction of carpule container 44 of FIGS. 10 and 11;

FIG. 13 is a three-dimensional depiction of a first component 68 that serves for mounting carpule container 44 on housing 34 and that here carries three ramps that serve to assist priming;

FIG. 14 is a three-dimensional depiction of component 68 of FIG. 13, but viewed from the side of the ramps;

FIG. 15 is a plan view of component 68 of FIGS. 13 and 14, viewed in the direction of arrow XV of FIG. 14;

FIG. 16 is a three-dimensional depiction of a second component 92 that likewise carries three ramps which interact with the ramps of the component of FIGS. 13 to 15;

FIG. 17 is a plan view from below of second component 92, viewed in the direction of arrow XVII of FIG. 18;

FIG. 18 is a side view looking in the direction of arrow XVIII of FIG. 17;

FIG. 19 is a plan view of component 92 of FIG. 16, viewed in the direction of arrow XIX of FIGS. 16 and 18;

FIG. 20 is a three-dimensional depiction of second component 92 in accordance with FIGS. 16 to 19, and of a compression spring acting on said component;

FIG. 21 is a depiction analogous to FIG. 20 but in a side view; the depiction of second component 92 corresponds approximately to the depiction in accordance with FIG. 18;

FIG. 22 is a plan view of housing 34, viewed in the direction of arrow XXII of FIG. 21;

FIG. 23 is an exploded view to explain the interaction of the parts depicted in the preceding figures;

FIG. 24 is a three-dimensional depiction analogous to FIG. 23;

FIG. 25 is a depiction which serves to explain the presetting of piston rod 38 (FIG. 9) against piston 48 of a carpule 50, and the simultaneous locking of second component 92 to prevent rotation;

FIG. 26 is a depiction showing the locked state of second component 92, as a result of which piston rod 38 (FIG. 9) is also blocked in terms of twisting but can be moved in an axial direction;

FIG. 27 is a depiction showing release of the locking of second component 92 before a carpule change, carpule container 44 being rotated in this context in the direction of arrow 58 as depicted;

FIG. 28 is a longitudinal section viewed in the direction of arrows XXVIII-XXVIII of FIG. 25;

FIG. 29 is a longitudinal section viewed in the direction of arrows XXIX-XXIX of FIG. 26;

FIG. 30 is a plan view of housing 34, viewed in the direction of arrow XXX of FIG. 31;

FIG. 31 is an exploded view of a simplified embodiment of the injection device;

FIG. 32 is a depiction analogous to FIG. 31 but as a three-dimensional view;

FIG. 33 shows a variant of FIG. 16 (of the embodiment according to FIGS. 16 to 29) which seems particularly suitable for injection devices in which a replacement of carpules is not possible, namely a component having three ramps 124 that interact with the ramps of the component of FIGS. 13 to 15;

FIG. 34 is a plan view from below of second component 92, viewed in the direction of arrow XXXIV of FIG. 35;

FIG. 35 is a side view looking in the direction of arrow XXXV of FIG. 34;

FIG. 36 is a plan view of the component of FIGS. 33 to 35, viewed in the direction of arrow XXXVI of FIG. 35;

FIG. 37 shows a variant of second component 92, having a thread pitch of its ramps 124′ that is reduced as compared with FIG. 35;

FIG. 38 is a plan view of part 92, viewed in the direction of arrow XXXVIII of FIG. 37;

FIG. 39 is a highly schematic depiction of parts of the injection device that are important here in the “presetting” procedure after a carpule change;

FIG. 40 shows the completion of presetting, plate 46′ of piston rod 38 abutting against piston 48, and piston rod 38 being blocked in terms of rotation; and

FIG. 41 shows a procedure in the context of carpule changing, in which carpule container 44 is rotated in the direction of an arrow 58, in order to move plate 46′ downward.

### DETAILED DESCRIPTION

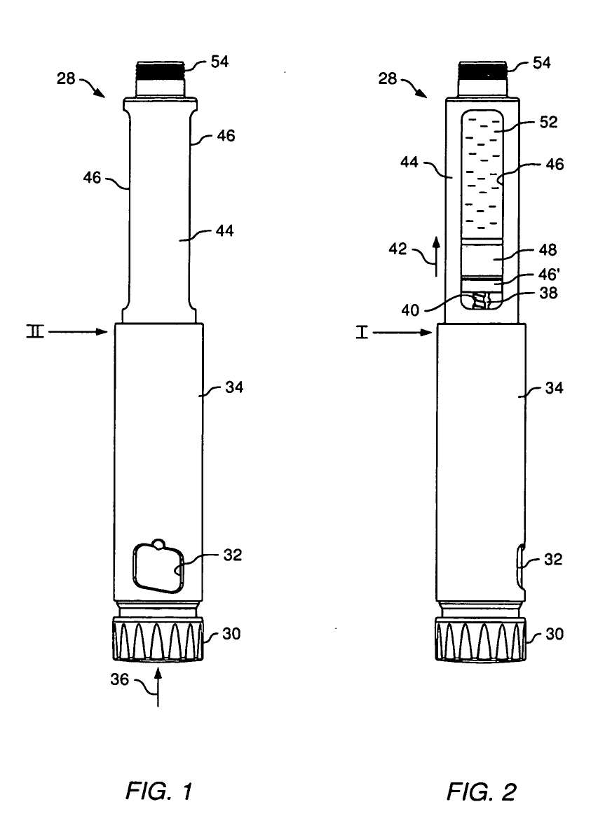

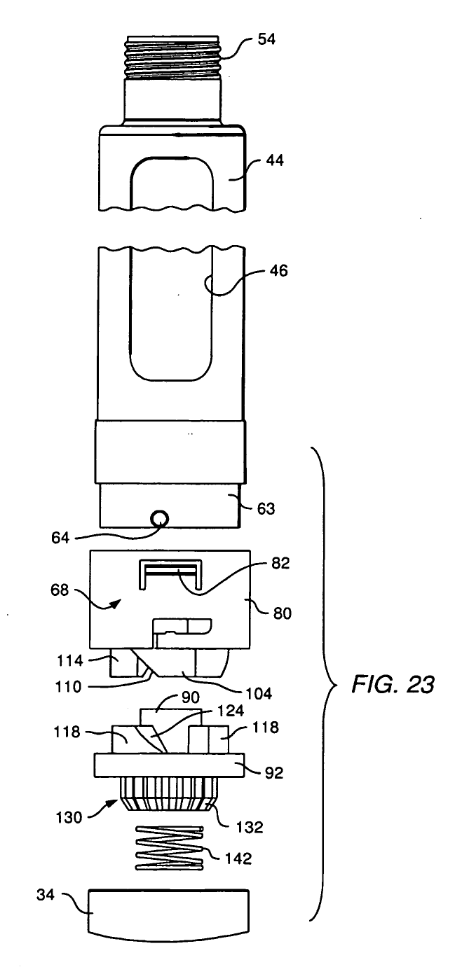

FIGS. 1 and 2 show an injection device 28 that, because of its small size, is also referred to as a “pen injector.” At the rear, i.e. at its end facing away from the patient, it has an adjusting knob 30 for setting a desired injection dose (by rotating knob 30), the dose that is set being displayed in a window 32. During setting, knob 30 is rotated out of housing 34, and during an injection the patient pushes on knob 30 in the direction of an arrow 36, i.e. toward the patient. The result is that a piston rod 38, which is equipped with an external thread 40, is moved forward in the direction of an arrow 42 toward the patient (who is to be thought of as being at the top in FIGS. 1 to 12 and 23 to 32). External thread 40 is depicted as a left-hand thread.

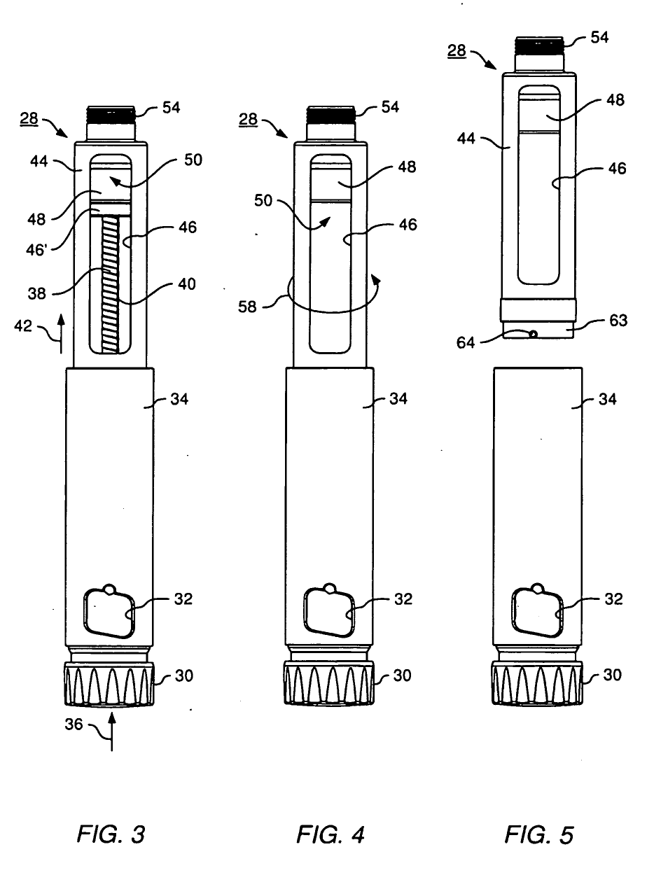

In FIGS. 2 and 3, piston rod 38 is located in a carpule container 44 that is equipped with two oppositely located windows 46.

FIG. 3 shows pen injector 28 with an empty carpule 50 whose shape can best be gathered from FIG. 11. The injection fluid 52 (FIGS. 2, 6, 7, 8, 9, 11) is exhausted, and piston rod 38 is in its maximally forward position. Before a completely filled carpule 50 can be inserted, as shown by FIG. 11, piston rod 38 must now be brought into its maximally rearward position.

In many pen injectors this is done by unscrewing carpule container 44 and then turning piston rod 38 back by turning a separate component that is referred to as a “return ring.” In other pen injectors piston rod 38 is simply pushed manually into housing 34 after carpule container 44 is removed.

In the case of the injection device depicted, piston rod 38 is screwed back by rotating carpule container 44, in this case by turning it counterclockwise (see arrow 58 in FIG. 4). (The rotation direction is indicated from the viewpoint of the front end of injector 28, i.e. as viewed from above.)

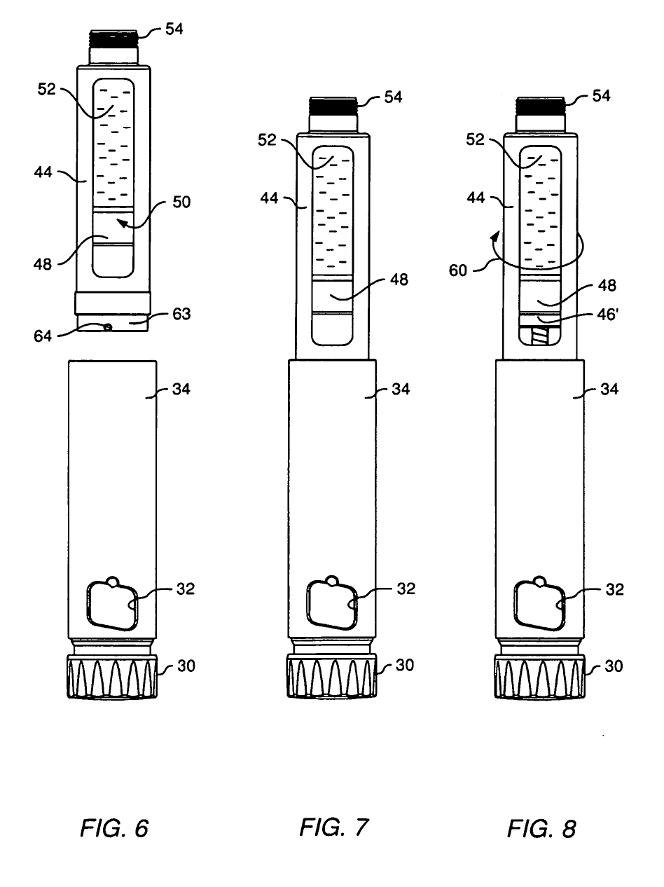

When piston rod 38 has reached its maximally rearward position, carpule container 44 can be removed (FIG. 5) and the empty carpule 50 can be replaced by a full one (see FIG. 6). Carpule container 44 can then be remounted onto housing 34 (see FIG. 7). Piston rod 38 is screwed forward by a rotation opposite to direction 58 (FIG. 4), i.e. in this case by a clockwise rotation (see arrow 60 of FIG. 8).

As soon as plate 46′ of piston rod 38 reaches piston 48 (see FIG. 8), the system automatically locks, i.e. a further rotation of carpule container 44 in a clockwise direction 60 becomes impossible. An elevated torque must now be exerted in order to rotate the carpule container counterclockwise (see arrow 58 of FIG. 4).

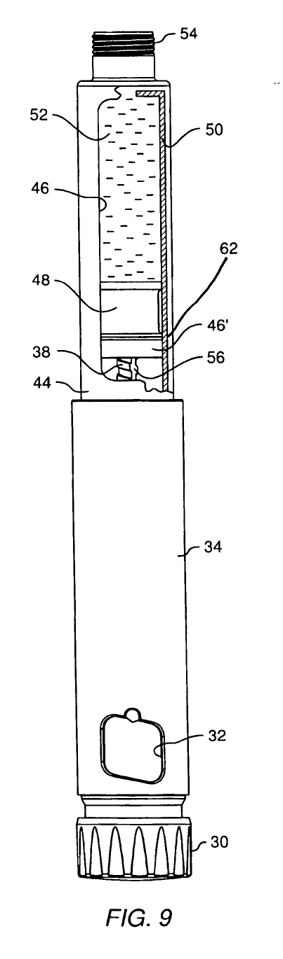

This prevents the patient from inadvertently rotating carpule container 44 counterclockwise even though he or she still wishes to withdraw injections from carpule 50 that is presently inserted. The reason is that a gap 62 (FIG. 9) would thereby be produced between plate 46′ and piston 48, so that the distance over which the latter would be moved forward during a subsequent injection, by plate 46′ of piston rod 38, is too small by an amount equal to the size of said gap 62, so that the fluid quantity injected in the context of the injection would correspondingly be too small. Correct “presetting” of plate 46′ against piston 48 is therefore very important.

In the locked state (FIG. 8), carpule container 44 can therefore be rotated counterclockwise only with an elevated torque. This provides protection against inadvertent initiation of a carpule change (FIG. 4), similar in effect to the protection that is usual with comparable pen injectors.

Once carpule container 44 is locked, as will be described below, the patient can begin with injections without needing to specifically prime the pen injector again. This results in very intuitive and easily understandable operation.

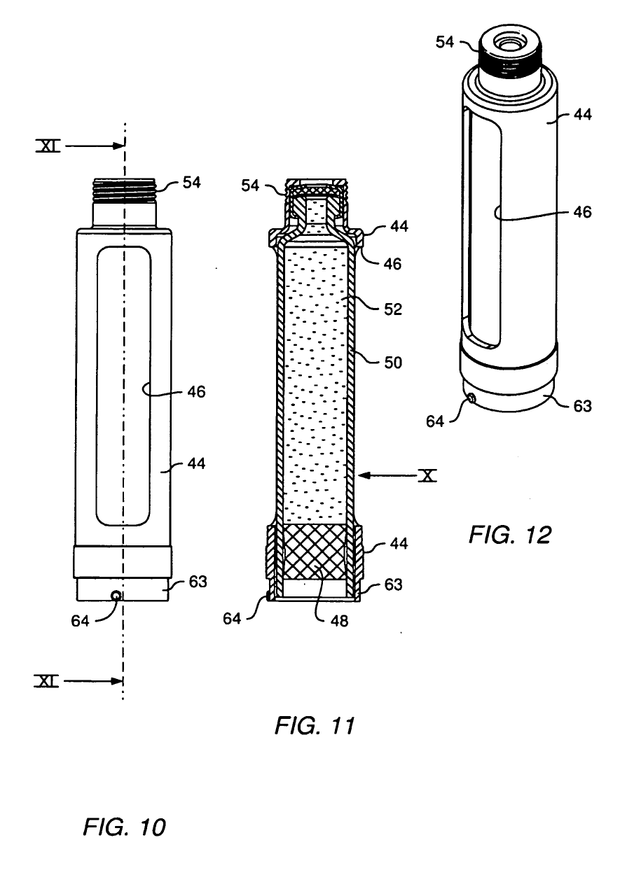

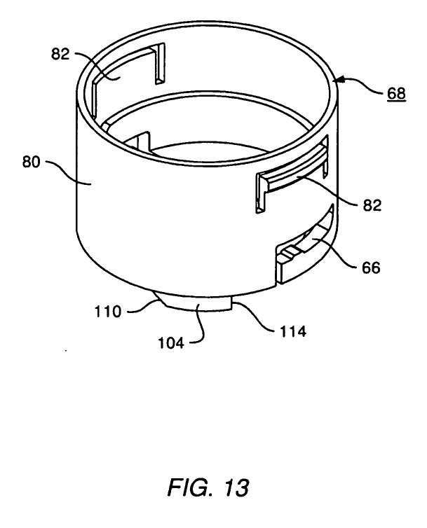

FIGS. 10 to 12 serve to explain carpule container 44. The latter has at its end 63 remote from the patient a peg 64 that serves for bayonet connection with a corresponding opening 66 of a first component 68 that is depicted in FIGS. 13 to 15 and 23 to 32.

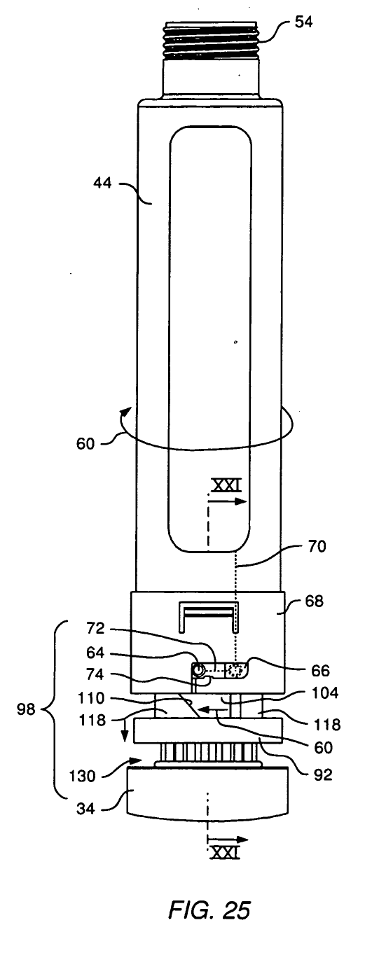

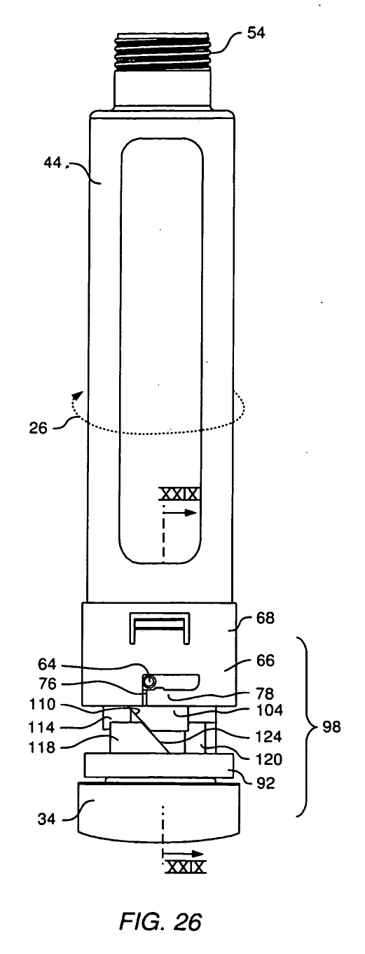

For latching (as shown in FIG. 25), peg 64 is introduced from above along a track 70 into opening 66, and then brought, by rotation (to the left) over a latching lug 74 along a distance 72, into the latched position depicted in FIGS. 25, 26, and 27. Lower boundary 78 (FIG. 26) of opening 66 is elastically resilient as a result of an axially extending opening 76 (FIG. 26), in order to enable a latching connection.

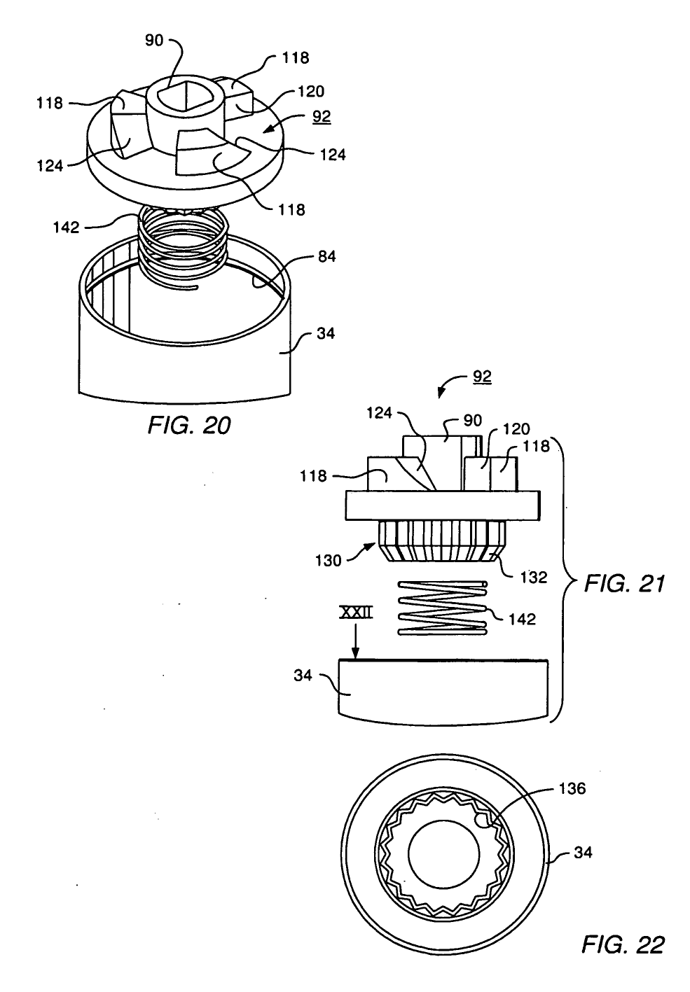

Component 68 has a hollow-cylindrical outer wall 80, and recessed into said wall are elastically resilient guidance members 82 that have associated with them, in housing 34, an annular groove 84 (FIGS. 20, 24, 28, 29). Upon assembly, guidance members 82 latch into this annular groove 84, and component 68 is then rotatably guided by guidance members 82 in annular groove 84 of housing 34 but cannot be displaced axially relative to housing 34. Component 68 is in turn connected fixedly, but disengageably, to carpule container 44 via bayonet connection 64, 66.

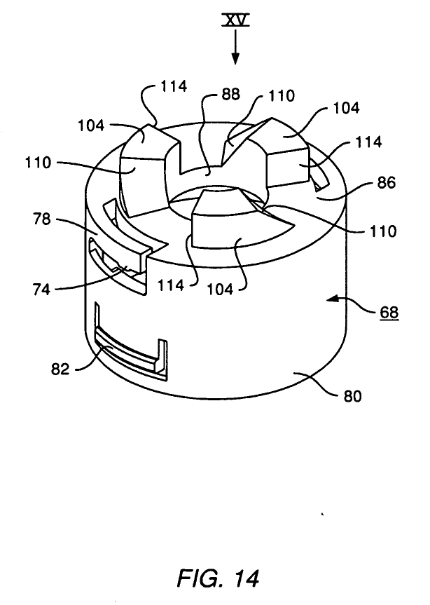

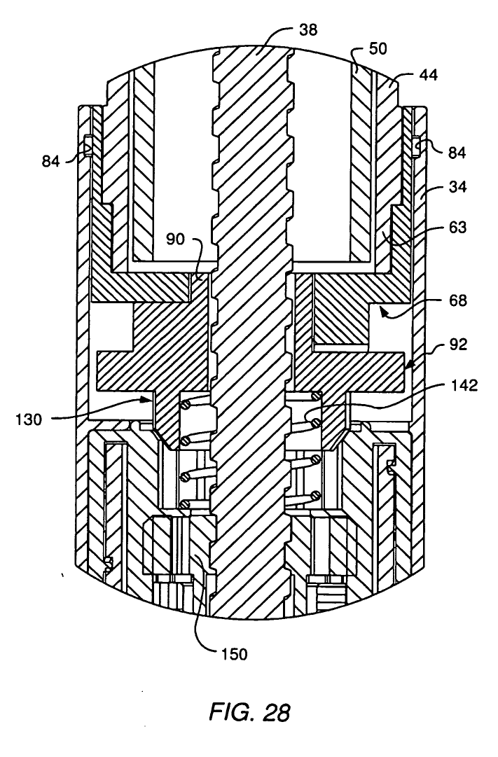

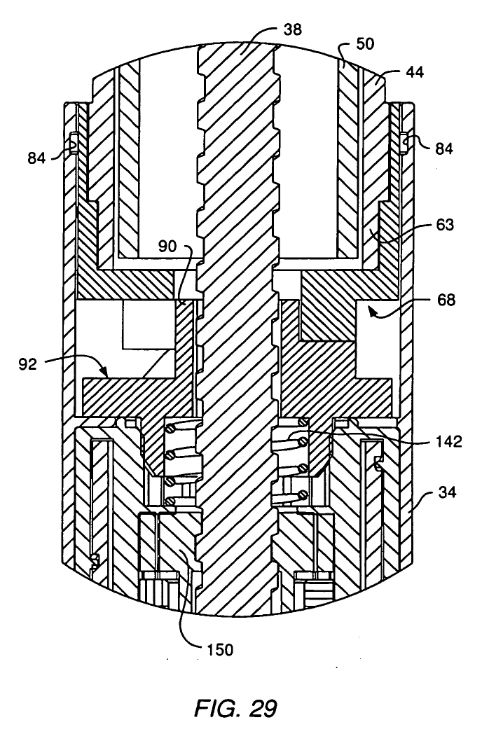

As FIG. 14 shows, component 68 has, adjacently to the cylindrical outer wall 80, a base 86 at whose center is located an opening 88 into which projects (as shown in FIGS. 28, 29) a collar 90 of a second component 92, so that components 68 and 92 are rotatable and also axially displaceable relative to one another.

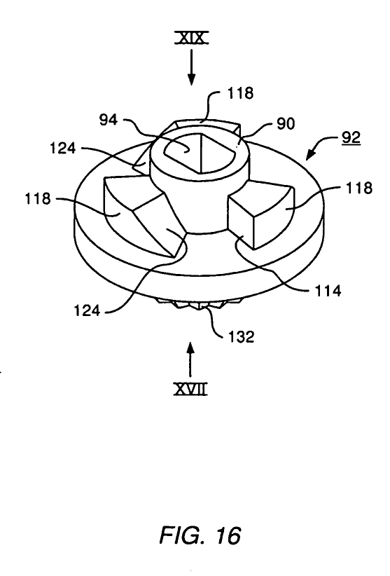

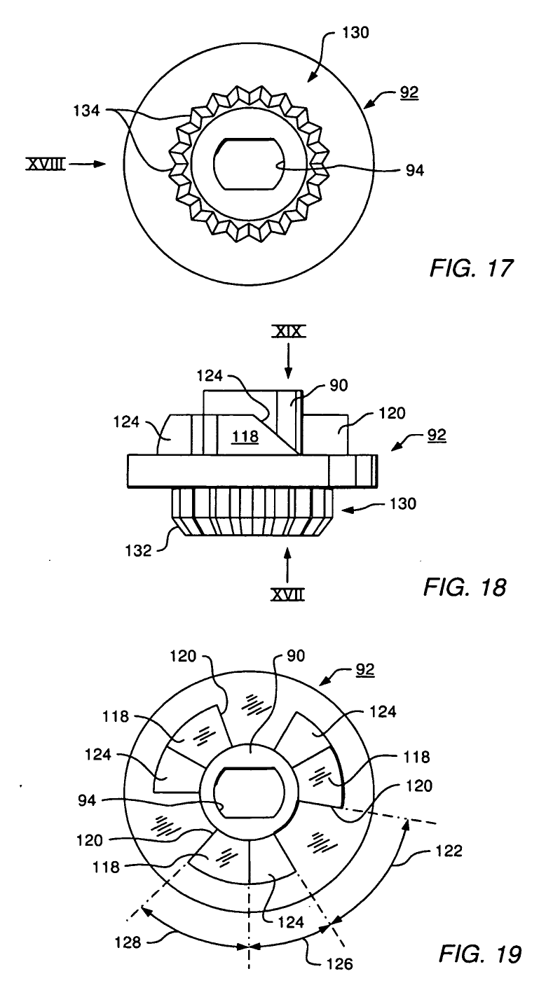

Collar 90 has an opening 94, extending in an axial direction, that serves for axial guidance of piston rod 38 and is therefore adapted to the latter's cross-sectional shape (as is shown, for example, by FIGS. 16, 17, and 19) so that piston rod 38 and second component 92 can rotate only together, but can shift axially relative to one another.

In contrast thereto, first component 68 can rotate relative to housing 34 but cannot shift axially. The same is then true of carpule container 44 when it is latched in on part 68.

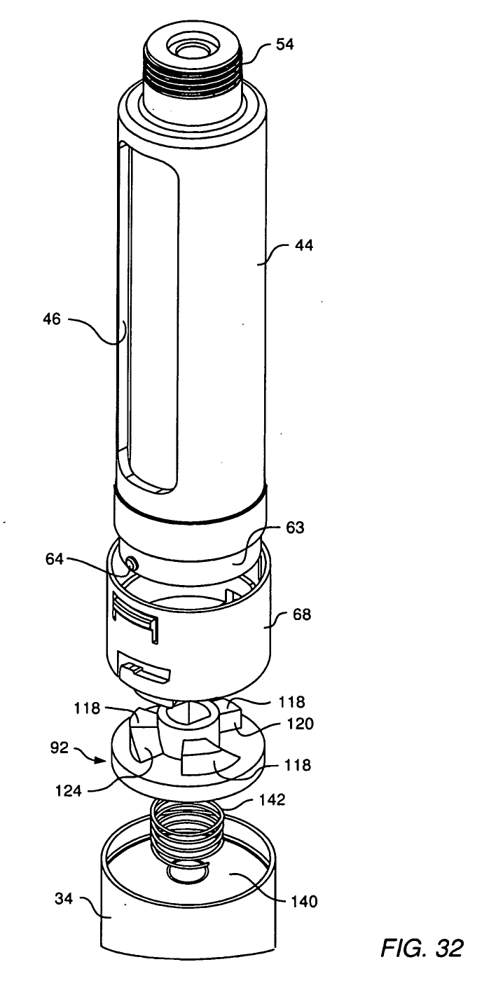

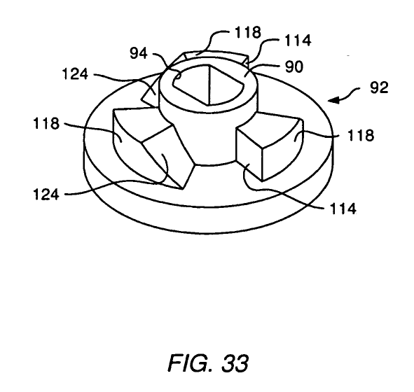

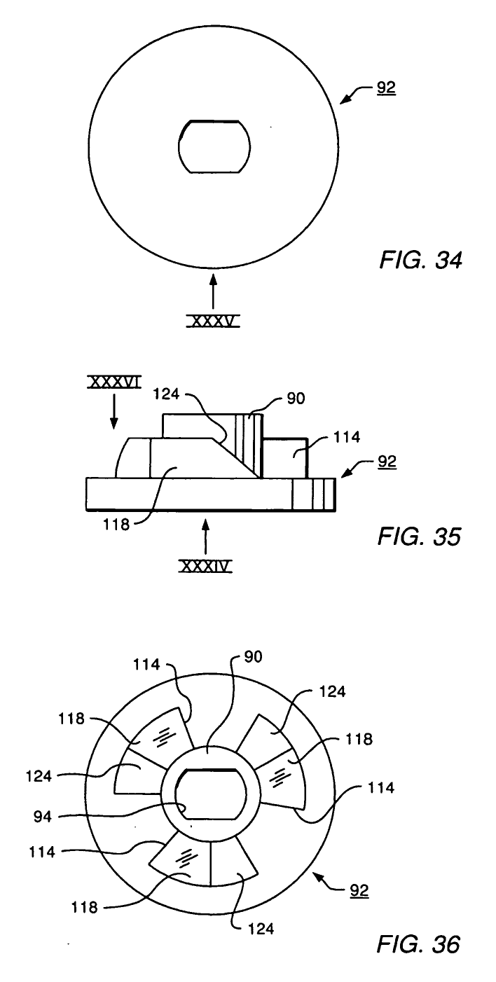

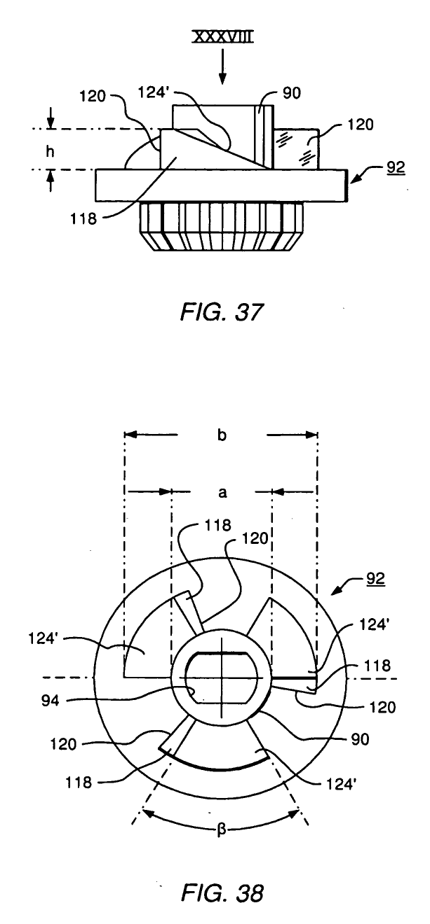

First component 68 and second component 92 together form a linkage 98 whose function will be described below with reference to FIGS. 25 to 27. It serves to convert a relative rotation between components 68 and 92 into an axial motion of second component 92, which motion has the function of immobilizing second component 92 and piston rod 38 guided therein, for example by positive engagement of component 92 with housing 34 (see FIGS. 21 to 23 and 25 to 27) or by generating a strong friction between second component 92 and housing 34 (as depicted in FIGS. 32 to 36).

As FIGS. 13 to 36 show, first component 68 and second component 92 are equipped with ramps 104 and 118, respectively.

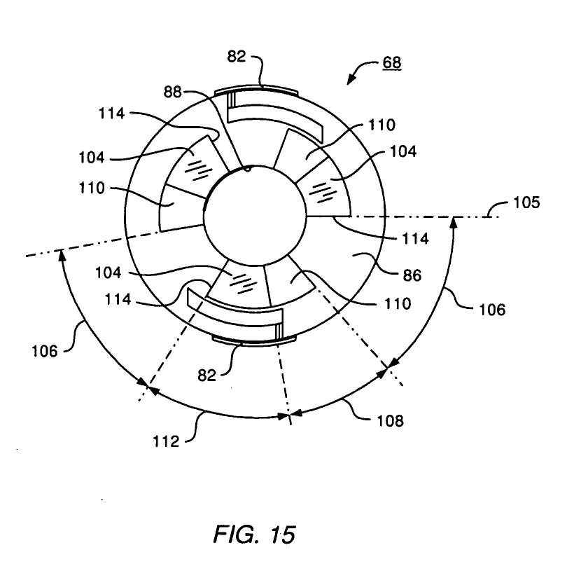

FIGS. 14 and 15 show, by way of example, three ramps 104 that are arranged on base 86 of first component 68 at equal spacings of 120°.

Proceeding, in FIG. 15, from a point 105 that would correspond on a clock to approximately three o'clock, this is followed clockwise firstly by a ramp-free portion 106 having an angular extent of approximately 50°. This is followed by a portion 108 (e.g. 30°) having a ramp portion 110 that usually rises to a maximum in portion 108.

There then follows a flat portion 112 (e.g. 40°) in which the height of ramp 104 does not substantially change further, and at the end 114 of this portion 112 the height of ramp 104 drops abruptly to zero, i.e. point 114 represents a shoulder of ramp 104. First component 68 thus has a total of three ramps 104, three shoulders 114, and three ramp portions 110.

The above-described configuration repeats after shoulder 114, i.e. the next ramp portion 110 begins to rise at an angular spacing 106 from shoulder 114, as is clearly evident from FIGS. 14 and 15.

Second component 92 (FIGS. 16 to 19) has a configuration largely complementary thereto, as shown by a comparison of FIGS. 15 and 19. It likewise has three ramps 118.

Beginning at a shoulder 120 (FIG. 19) at a location corresponding approximately to four o'clock there comes first (viewed clockwise) a flat region 122 (e.g. 50°) adjacent to which is a rising region 124 of the lower (in FIG. 19) ramp 118. This ramp region 124 has in this example an angular extent 126 of approximately 30°, and it ends in a flat roof region having an angular extent 128 of, for example, 40°, at the end of which a shoulder 120 is again located.

Ramps 118 are located around collar 90, and located inside collar 90 is opening 94 in which piston rod 38 is guided. When second component 92 rotates, piston rod 38 therefore also rotates, and the latter can shift freely in an axial direction in opening 94 as is necessary, for example, when priming.

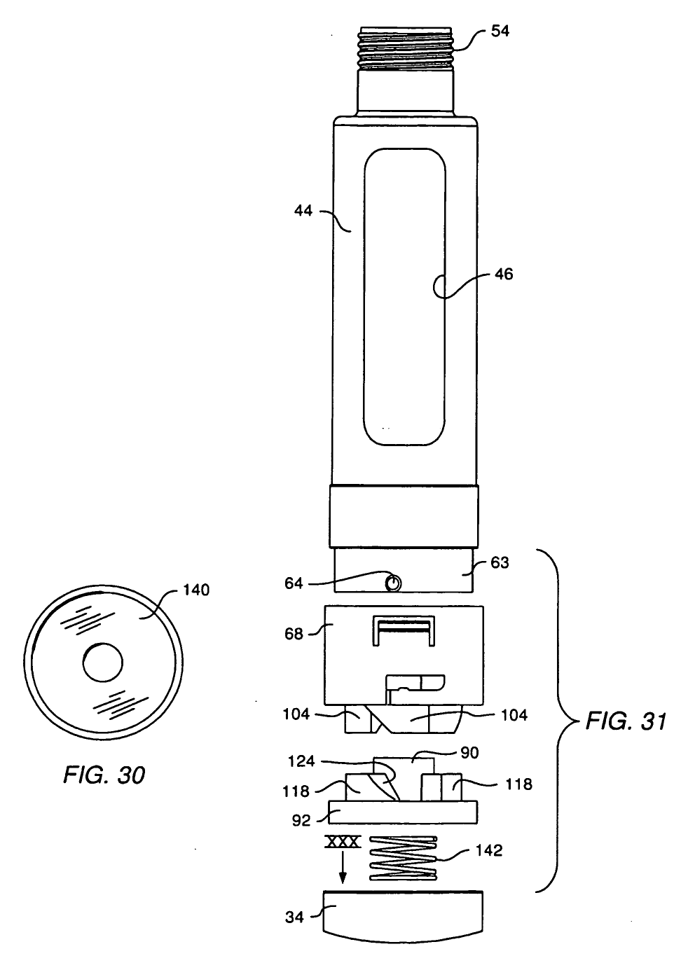

On its side facing away from ramps 118, second component 92 has a coupling projection 130 that tapers frustoconically at its free end 132 and is equipped with longitudinal grooves 134 for engagement into corresponding longitudinal grooves 136 of housing 34, so that projection 130, upon engagement into longitudinal grooves 136, is prevented by positive engagement from rotating. This effect can optionally also, in accordance with FIGS. 30 to 36, be achieved without the projection by the fact that second component 92 is simply pressed against a surface 140 (FIG. 32) of housing 34 and secured there by friction to prevent rotation.

Second component 92 is pressed by a spring 142 in a direction toward first component 68, which latter is guided in the housing rotatably, but (because of guidance members 82 and annular groove 84) axially nondisplaceably.

### Mode of Operation

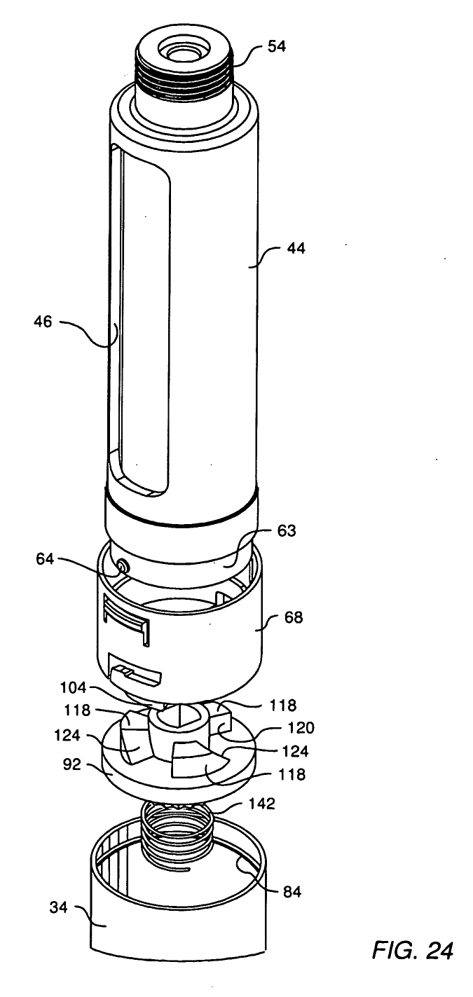

As FIG. 25 shows, upon a clockwise rotation 60 such as that which occurs after insertion of a new carpule 50 (see FIG. 8), ramps 104 of first component 68 are located between ramps 118 of second component 92, since the latter is pressed by spring 142 (FIG. 24) against first component 68. As a result, coupling part 130 is out of engagement with coupling part 136 provided in housing 34, so that when the patient rotates carpule container 44 clockwise (arrow 60), first component 68 with its ramps 104 also rotates along with carpule container 44, and said ramps 104 engage (as shown in FIG. 25) between ramps 118 of second component 92 and thereby also transfer said rotary motion 60 to second component 92 and to piston rod 38 guided therein.

The latter is guided in housing 34 in a threaded part 150 (FIGS. 28, 29) that, for example, can be part of the dosing apparatus of the injector and that does not rotate during a carpule change. Piston rod 38 is therefore moved in an upward direction in FIG. 28, i.e. toward the patient, in the context of a rotation 60 (FIG. 25).

As depicted in FIG. 9, piston rod 38 thereby comes into contact by means of its plate 46′ against piston 48 in carpule 50, i.e. injector 28 is now primed, i.e. correctly prepared for an injection.

Piston rod 38 therefore cannot move any farther upward, i.e. the torque in the direction of arrow 60 (FIG. 25) continues to act because the patient is continuing to turn carpule container 44 as depicted in FIG. 26, but the rotation of second component 92 is now blocked because piston rod 38 is abutting against piston 48 (see FIG. 8).

Oblique surfaces 110 (FIGS. 14 to 16) of ramps 104 of first component 68 therefore now produce an axial force on oblique surfaces 124 of ramps 118 of second component 92 and displace the latter, as depicted in FIG. 26, against the force of spring 142 (FIG. 21) away from first component 68. This results in a coupling between projection 130 (FIG. 21) and longitudinal grooves 136 in housing 34 (FIG. 22), so that second component 92 cannot rotate any farther in a clockwise direction 60.

Injection device 28 is thus now ready to use, i.e. the patient can, in normal fashion, set his or her individual injection dose and give him- or herself injections until the contents of carpule 50 are exhausted.

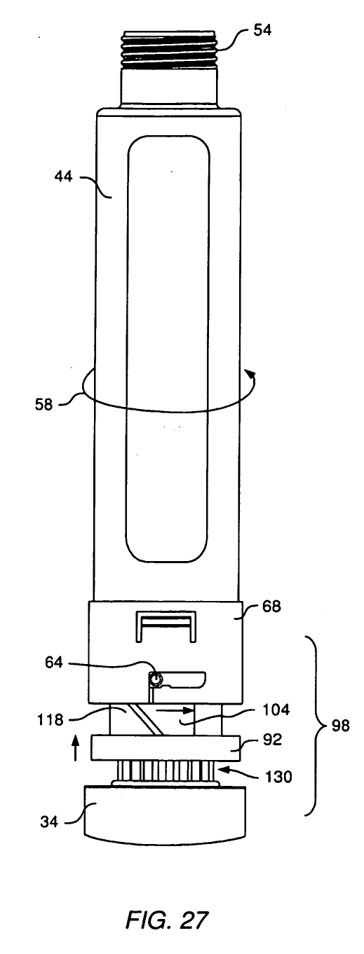

Carpule container 44 must then be removed. For this, it is rotated counterclockwise in direction 58, as shown in FIG. 27. This causes the previous coupling between first component 68 and second component 92 to disengage; the latter is displaced upward by spring 142; and shoulders 114 of first component 68 come into abutment against shoulders 120 of second component 92 so that the latter is likewise driven in counterclockwise direction 58.

As a result, piston rod 38 is rotated into housing 34 until it comes to a stop, and at the end of this procedure bayonet closure 64, 66 is disengaged, so that carpule container 44 can be taken off in order to remove the exhausted carpule and insert a new carpule 50, as has already been described with reference to FIGS. 1 to 9.

The above-described procedure is then repeated in order to prime the new carpule 50 again, and once again prepare injection device 28 for reliable use.

In order to optimize the present invention, the slope of the ramps was also modified.

Each of these ramps can be imagined as part of a thread whose (notional) flights have a specific pitch. This thread pitch is the spacing from one flight to the next, and in this case is preferably approximately 10 to 20 mm.

The inside diameter a and outside diameter b of ramps 124′ of part 92 are plotted in FIG. 38. Practical values can be, for example,
a=6 mm
b=11.5 mm

One ramp 124′ extends here over an angle beta that is equal, for example, to 40°.

If parts 68 and 92 rotate during presetting through an angle of 36° relative to one another, their spacing h then changes by a value

h=360°/36°*thread pitch (1).

If the thread pitch is equal to 15 mm, h therefore has a value of approximately 1.5 mm, which experiments have shown to be a favorable value.

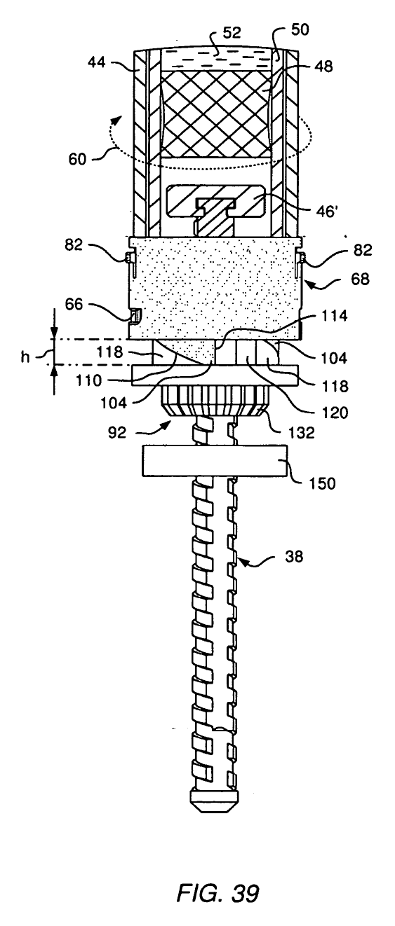

Ramps 104 on part 68 have the same shape as ramps 124′ of part 92, and are therefore shown only in FIGS. 39 to 41. Part 68 and its ramps 104 are shown therein in gray in order to facilitate comprehension.

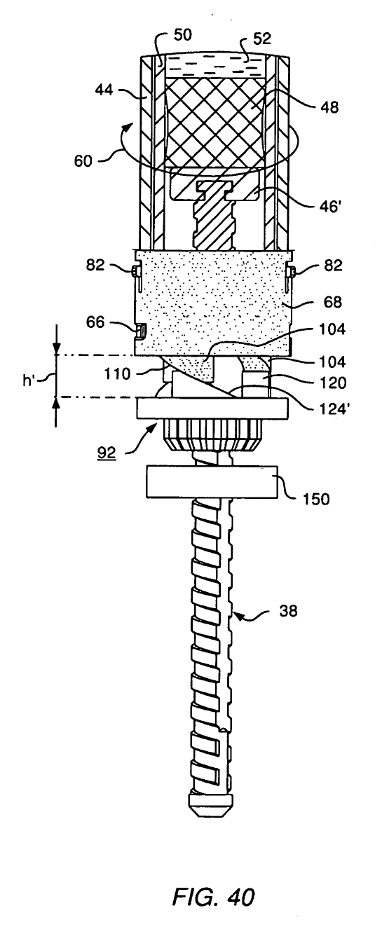

FIGS. 39 to 41 are highly schematic depictions of the adjusting procedures. FIG. 39 shows the presetting of plate 46′ against piston 48. Ramps 118 are rotated in the direction of arrow 60 so that they initially move plate 46′ upward toward piston 48. In FIG. 40, plate 46′ has reached piston 48. Upper ramps 110 therefore now shift relative to lower ramps 124′ and displace part 92 downward, so that the latter is now at a spacing h′ from part 68, and part 92 is blocked in terms of rotation in the manner described. The injection device is now primed.

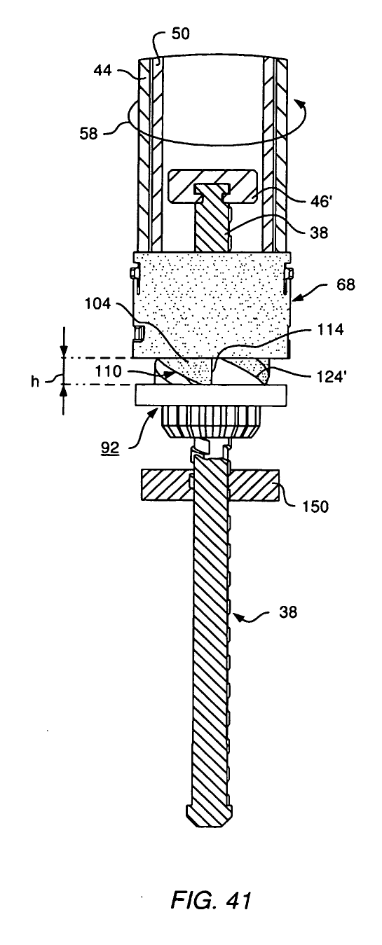

FIG. 41 shows the procedure in the context of a cartridge change. Carpule container 44 is rotated in the direction of arrow 58, with the result that ramps 104 disengage from ramps 124′ and the spacing between parts 68 and 92 once again becomes h. Part 92 is pressed by its spring 142 (see FIG. 31) toward part 68. Ramps 104 now press with their steep flanks 114 against the corresponding flanks 120 (FIG. 40) of ramps 110.

Because part 68 is rotating together with part 92 in the direction of arrow 58, plate 46′ is moved downward; and at the end of this movement, part 68 disengages from dosing part 44, as depicted in FIG. 5. Operation is therefore very simple and intuitive, and dosing accuracy is correspondingly increased.

Numerous variants and modifications are, of course, possible within the scope of the present invention.

What is claimed is:

1. An injection device,
having a housing (34),
and having a carpule container (44), associated with said housing (34), for receiving a carpule (50) having a fluid (52) to be injected,
and having a piston (48) displaceable in said carpule (50), and having a piston rod (38) which serves, in the context of an injection, to displace the piston (48) of a carpule (50) inserted into the carpule container (44) and thereby to eject fluid (52) from the carpule (50),
which piston rod (38) comprises an external thread (40) that is in engagement with an internal thread formed on a component (150) arranged in the housing (34),
further having a linkage (98) that comprises a first component (68) which is arranged rotatably but axially nondisplaceably relative to the housing (34) and is connected to the carpule container (44) during operation,
and a second component (92) which is connected nonrotatably but axially displaceably to the piston rod (38),
so that the first component (68), the second component (92), and the piston rod (38) can together rotate relative to the housing (34) as long as the piston rod (38) is not impeded from being displaced axially,
and so that, when the piston rod (38) is impeded by the piston (48) of a carpule (58) from being further displaced toward said piston (48), the first component (68) and the second component (92) move relative to one another and thereby displace the second component (92) into a position in which the possibility of a rotation of the second component (92), and of the piston rod (38) connected nonrotatably to it, relative to the housing (34) is at least impeded.

2. The injection device according to claim 1, wherein
the linkage (98) has a first ramp (104) that is provided on that side of the first component (68) which faces toward the second component (92),
and a second ramp (118) that is provided on that side of the second component (92) which faces toward the first component (68),
and which interacts with the first ramp (104),
in order, upon a relative rotation between the first component (68) and the second component (92), to modify an axial spacing (h) between the first component (69) and the second component (92) and to thereby displace the second component (92) into the position in which its rotation relative to the housing (34) is at least impeded.

3. The injection device according to claim 2, wherein
at least one of the ramps (104, 118) transitions into a portion that extends approximately perpendicular to a rotation axis of the first component (68), in order to prevent a change in the axial spacing (h) between the first component (68) and the second component (92) in a predefined range of the relative rotation between the first component (68) and second component (92).

4. The injection device according to claim 1, wherein
a spring element (142) that biases the second component (92) toward the first component (68) is provided.

5. The injection device according to claim 1, wherein
the second component (92) is implemented as a return member for returning the piston rod (38) before a change of the carpule (50).

6. The injection device according to claim 1, wherein
the first component (68) is implemented (64, 66), on its side facing toward the carpule container (44), for disengageable connection with the carpule container (44).

7. The injection device according to claim 1, wherein
the first component (68) and the second component (92) comprise with respect to each other a bearing capability (88, 90) that enables them to rotate relative to one another.

8. The injection device according to claim 1, wherein
the linkage (98) is so implemented that when, as a result of a rotation, occurring relative to the housing (34), of the first component (68), the second component (92), and the piston rod (38) together,
the latter has reached the piston (48) of a carpule (50) inserted into the carpule container (44), the second component (92) is displaceable into a locking position in which an engagement part (130) provided on the second component (92) engages into a countermember (134) connected nonrotatably to the housing (34), and
thereby blocks a further rotation of the second component (92) and of the piston rod (38) connected nonrotatably thereto.

9. The injection device according to claim 2, wherein
the ramps (104, 118) are each implemented as a (notional) thread flight.

10. The injection device according to claim 9, wherein
the thread pitch, i.e. the spacing between two adjacent flights, has a value in a range from approximately 10 mm to approximately 20 mm.

11. The injection device according to claim 10, wherein
the thread pitch has a value in a range from approximately 14 mm to approximately 16 mm.

12. The injection device according to claim 2, wherein
three ramps are provided respectively on the first component (68) and on the second component (92), the ramps of the first component (68) interacting at least partly with the ramps of the second component (92).

13. The injection device according to claim 3, wherein
three ramps are provided respectively on the first component (68) and on the second component (92), the ramps of the first component (68) interacting at least partly with the ramps of the second component (92).

14. The injection device according to claim 9, wherein
three ramps are provided respectively on the first component (68) and on the second component (92), the ramps of the first component (68) interacting at least partly with the ramps of the second component (92).

15. The injection device according to claim 10, wherein
three ramps are provided respectively on the first component (68) and on the second component (92), the ramps of the first component (68) interacting at least partly with the ramps of the second component (92).

16. The injection device according to claim 11, wherein
three ramps are provided respectively on the first component (68) and on the second component (92), the ramps of the first component (68) interacting at least partly with the ramps of the second component (92).

17. The injection device according to claim 2, wherein
the linkage (98) is so implemented that when, as a result of a rotation, occurring relative to the housing (34), of the first component (68), the second component (92), and the piston rod (38) together,
the latter has reached the piston (48) of a carpule (50) inserted into the carpule container (44), the second component (92) is displaceable into a locking position in which an engagement part (130) provided on the second component (92) engages into a countermember (134) connected nonrotatably to the housing (34), and
thereby blocks a further rotation of the second component (92) and of the piston rod (38) connected nonrotatably thereto.

18. The injection device according to claim 3, wherein
the linkage (98) is so implemented that when, as a result of a rotation, occurring relative to the housing (34), of the first component (68), the second component (92), and the piston rod (38) together,
the latter has reached the piston (48) of a carpule (50) inserted into the carpule container (44), the second component (92) is displaceable into a locking position in which an engagement part (130) provided on the second component (92) engages into a countermember (134) connected nonrotatably to the housing (34), and
thereby blocks a further rotation of the second component (92) and of the piston rod (38) connected nonrotatably thereto.

19. The injection device according to claim 4, wherein
the linkage (98) is so implemented that when, as a result of a rotation, occurring relative to the housing (34), of the first component (68), the second component (92), and the piston rod (38) together,
the latter has reached the piston (48) of a carpule (50) inserted into the carpule container (44), the second component (92) is displaceable into a locking position in which an engagement part (130) provided on the second component (92) engages into a countermember (134) connected nonrotatably to the housing (34), and
thereby blocks a further rotation of the second component (92) and of the piston rod (38) connected nonrotatably thereto.
